(19) United States
(12) Patent Application Publication
(10) Pub. No.: US 2023/0181837 A1
(43) Pub. Date: Jun. 15, 2023

(54) Supplemental Device for an Injection Device

(71) Applicant: Sanofi, Paris (FR)

(72) Inventors: Adam Moyo Harvey-Cook, Essex (GB); David Aubrey Plumptre, Worcestershire (GB); Oliver Charles Gazeley, Basel (CH); Robert Veasey, Warwick, Warwickshire (GB); Aidan Michael O`Hare, Warwick, Warwickshire (GB)

(21) Appl. No.: 17/921,276

(22) PCT Filed: May 5, 2021
(86) PCT No.: PCT/EP2021/061773

§ 371 Date: Oct. 25, 2022

(30) Foreign Application Priority Data
May 6, 2020 (EP) 20315234.3

(51) Int. Cl. A61M 5/315

(52) U.S. Cl. CPC A61M 5/31546; A61M 2205/587; A61M 2205/583; A61M 2205/50

(57) ABSTRACT

A supplemental device is configured to be coupled to an injection device. The supplemental device comprises a sensor arrangement and a processor arrangement. The processor arrangement is configured to cause the supplemental device to use the sensor arrangement to sense an identification feature of the injection device and to determine a type of the injection device based on the sensed identification feature.

### CROSS-REFERENCE TO RELATED APPLICATIONS

The present application is the national stage entry of International Patent Application No. PCT/EP2021/061773, filed on May 5, 2021, and claims priority to Application No. EP 20315234.3, filed on May 6, 2020, the disclosures of which are incorporated herein by reference.

### TECHNICAL FIELD

The present disclosure relates to a supplemental device for attachment to an injection device.

### BACKGROUND

A variety of diseases exists that require regular treatment by delivery, particularly injection of a medicament. Such injection can be performed by using injection devices, which are applied either by medical personnel or by patients themselves.

Drug injection devices particularly for usage by patients themselves may be equipped with electronics for measuring and storing data related to the usage. The usage related data may also be transmitted via a wireless link or a wireline connection to an external device such as a smartphone, a tablet or laptop computer, or in the cloud.

### SUMMARY

According to an aspect of the present disclosure, there is provided a supplemental device configured to be coupled to an injection device, the supplemental device comprising: a sensor arrangement; and a processor arrangement configured to cause the supplemental device to: use the sensor arrangement to sense an identification feature of the injection device; and determine a type of the injection device based on the sensed identification feature. This may allow the supplemental device to be used with more than one different type of injection device, with the supplemental device able to differentiate between different types of injection device to which it is attached. Operation of the supplemental device may be modified based on the type of the injection device determined by the supplemental device. This can allow the supplemental device to be more versatile in its operation. In other words, the supplemental device may determine a type of the injection device based on the sensed identification feature such that it can differentiate between different types of injection device and modify its operation accordingly.

The supplemental device may further comprise: a dose determination unit; wherein the processor arrangement is further configured to cause the supplemental device to: determine a dose using the dose determination unit, wherein the dose is determined based on the determined type of injection device.

The supplemental device may further comprise: an alignment arrangement, wherein the alignment arrangement is configured to interact with a corresponding alignment feature of the injection device such that the supplemental device can be coupled to the injection device in a predetermined number of orientations relative to the injection device, wherein the predetermined number is preferably one or two, or is three, four or more.

The supplemental device may further comprise: a communication interface, wherein the processor arrangement is configured to transmit data indicating the determined type of the injection device to an external computing device via the communication interface.

The sensor arrangement may comprise a light source and a color sensor, wherein the light source is arranged to illuminate a portion of the injection device when the supplemental device is coupled to the injection device, wherein the color sensor is arranged to determine a color of the illuminated portion, and wherein the processor arrangement is configured to determine the type of the injection device based on the determined color.

The sensor arrangement may comprise a navigation switch movable between a plurality of positions, wherein the navigation switch is arranged to be biased into a position of the plurality of positions by a biasing feature of the injection device when the supplemental device is coupled to the injection device, wherein each position of the plurality of positions corresponds to a type of injection device, and wherein the processor arrangement is configured to determine the type of the injection device based on the position of the navigation switch.

The sensor arrangement may comprise a plurality of contacts arranged to contact a conductive track formed on a surface of the injection device when the supplemental device is coupled to the injection device, wherein the conductive track comprises one or more segments arranged to electrically couple two or more contacts, wherein the processor arrangement is configured to determine the type of the injection device by polling each of the contacts.

The sensor arrangement may comprise two pins arranged to contact a conductive track formed on a surface of the injection device when the supplemental device is coupled to the injection device, wherein the processor arrangement is configured to determine the type of the injection device based on a determined resistance of the conductive track.

The sensor arrangement may comprise a magnetic field sensor arranged to detect a magnetic field generated by the injection device when the supplemental device is coupled to the injection device, wherein the processor arrangement is configured to determine the type of injection device based on the detected magnetic field.

The sensor arrangement may comprise at least one proximity sensor arranged to determine a distance between the supplemental device and a respective identification feature of the injection device when the supplemental device is coupled to the injection device, wherein the processor arrangement is configured to determine the type of the injection device based on the determined distance.

The proximity sensor may comprise an infrared transceiver, wherein the infrared transceiver is arranged to determine the depth of a hole arranged on a surface of the injection device when the supplemental device is coupled to the injection device, wherein the processor arrangement is configured to determine the type of the injection device based on the determined depth of the hole.

The sensor arrangement may comprise a first infrared sensor and a second infrared sensor, wherein the first infrared sensor and second infrared sensor are both arranged to read an encoded track formed on the injection device as the supplemental device is moved relative to the injection device during coupling of the supplemental device to the injection device, wherein the processor arrangement is configured to determine the type of the injection device based on the code read using the first infrared sensor and second infrared sensor.

The sensor arrangement may comprise a plurality of pins configured to form an electrical connection to a non-volatile memory in the injection device when the supplemental device is coupled to the injection device, wherein the processor arrangement is configured to read data from the non-volatile memory when the supplemental device is coupled to the injection device, and wherein the processor arrangement is configured to determine the type of the injection device based on the data read from the non-volatile memory.

According to another aspect of the present disclosure, there is provided a system comprising a supplemental device according to any preceding example and an injection device, wherein the injection device comprises an identification feature, and wherein the processor arrangement is configured to cause the supplemental device to:

use the sensor arrangement to sense the identification feature of the injection device; and

determine a type of the injection device based on the sensed identification feature.

According to another aspect of the present disclosure, there is provided a method of identifying a type of injection device using a supplemental device coupled to the injection device, the supplemental device comprising a sensor arrangement and a processor arrangement, the method comprising: causing, by the processor arrangement, the sensor arrangement to sense an identification feature of the injection device; and

determining, by the processor arrangement, a type of the injection device based on the sensed identification feature.

Aspects of the present disclosure may provide a supplemental device that can be coupled to more than one different type of injection device. The supplemental device may be able to differentiate between different types of injection device and modify its operation accordingly. The supplemental device may therefore have improved versatility. The identification feature on the injection device provides a simple means of allowing a type of the injection device to be identified by the supplemental device. Only a small modification to the injection device may be required to provide it with the identification feature.

### BRIEF DESCRIPTION OF THE FIGURES

Embodiments of the present disclosure will now be described, by way of example only, with reference to the accompanying drawings, in which:

FIG. 1 shows an exploded view of an injection device for use with a supplemental device according to embodiments of this disclosure;

FIG. 2 depicts a supplemental device according to embodiments of this disclosure, attached to the injection device of FIG. 1;

FIG. 3 is a block diagram of the supplemental device shown in FIG. 2;

FIG. 4A depicts cross-sectional view of the supplemental device shown in FIG. 2 according to a first embodiment of this disclosure;

FIG. 4B depicts a bottom view of the supplemental device of FIG. 4A;

FIG. 5A depicts cross-sectional view of the supplemental device shown in FIG. 2 according to a second embodiment of this disclosure;

FIG. 5B depicts a bottom view of the supplemental device of FIG. 5A;

FIG. 5C shows a portion of the injection device of FIG. 1 to which the supplemental device of FIG. 5A is to be coupled;

FIG. 6A depicts cross-sectional view of the supplemental device shown in FIG. 2 according to a third embodiment of this disclosure;

FIG. 6B depicts a bottom view of the supplemental device of FIG. 6A;

FIG. 6C shows a portion of the injection device of FIG. 1 to which the supplemental device of FIG. 6A is to be coupled;

FIG. 7A depicts cross-sectional view of the supplemental device shown in FIG. 2 according to a fourth embodiment of this disclosure;

FIG. 7B depicts a bottom view of the supplemental device of FIG. 7A;

FIG. 7C shows a portion of the injection device of FIG. 1 to which the supplemental device of FIG. 7A is to be coupled;

FIG. 8A depicts cross-sectional view of the supplemental device shown in FIG. 2 according to a fifth embodiment of this disclosure;

FIG. 8B depicts a bottom view of the supplemental device of FIG. 8A;

FIG. 8C shows a portion of the injection device of FIG. 1 to which the supplemental device of FIG. 8A is to be coupled;

FIG. 9A depicts cross-sectional view of the supplemental device shown in FIG. 2 according to a sixth embodiment of this disclosure;

FIG. 9B depicts a bottom view of the supplemental device of FIG. 9A;

FIG. 9C shows a portion of the injection device of FIG. 1 to which the supplemental device of FIG. 9A is to be coupled;

FIG. 9D is a schematic illustration of the supplemental device of FIG. 9A when coupled to the injection device of FIG. 9C;

FIG. 10A depicts cross-sectional view of the supplemental device shown in FIG. 2 according to a seventh embodiment of this disclosure;

FIG. 10B depicts a bottom view of the supplemental device of FIG. 10A;

FIG. 10C is a schematic illustration of the operation of the supplemental device of FIG. 10A;

FIG. 11A depicts cross-sectional view of the supplemental device shown in FIG. 2 according to an eighth embodiment of this disclosure;

FIG. 11B depicts a bottom view of the supplemental device of FIG. 11A;

FIG. 11C shows a portion of the injection device of FIG. 1 to which the supplemental device of FIG. 11A is to be coupled; and

FIG. 12 is a flow diagram illustrating a method according to the present disclosure.

### DETAILED DESCRIPTION

In the following, embodiments of the present disclosure may be described with reference to an insulin injection device. The present disclosure is however not limited to such application and may equally well be deployed with injection devices that eject other medicaments.

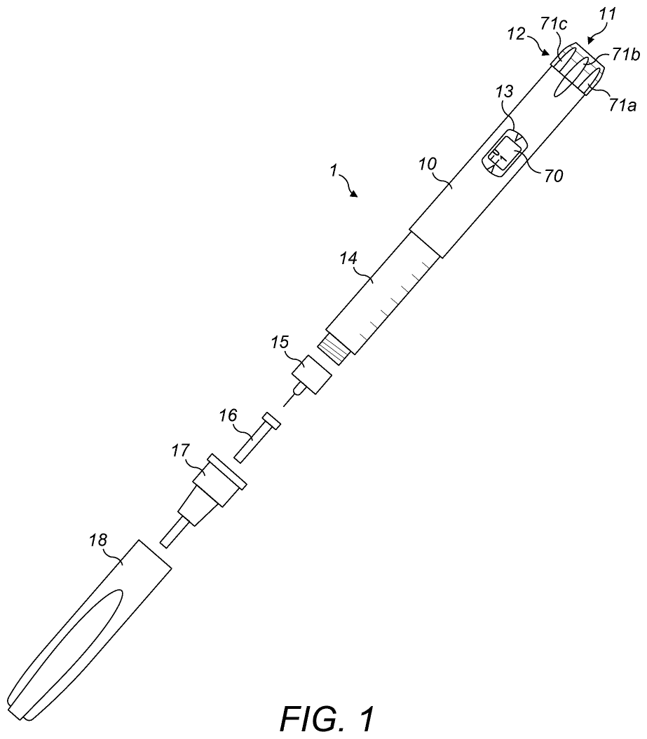

FIG. 1 is an exploded view of a medicament delivery device. In this example, the medicament delivery device is an injection device 1, such as Sanofi's SoloSTAR® insulin injection pen, but aspects of the present disclosure may apply to other types of injection device 1 and injection pen.

The injection device 1 of FIG. 1 is a pre-filled, disposable injection pen that comprises a housing 10 and contains an insulin container 14, to which a needle 15 can be affixed. The needle is protected by an inner needle cap 16 and either an outer needle cap 17 other cap 18. An insulin dose to be ejected from injection device 1 can be programmed, or ‘dialled in’ by turning a dosage knob 12, and a currently programmed dose is then displayed via dosage window 13, for instance in multiples of units. For example, where the injection device 1 is configured to administer human insulin, the dosage may be displayed in so-called International Units (IU), wherein one IU is the biological equivalent of about 45.5 micrograms of pure crystalline insulin (1/22 mg). Other units may be employed in injection devices for delivering analogue insulin or other medicaments. It should be noted that the selected dose may equally well be displayed differently than as shown in the dosage window 13 in FIG. 1.

The dosage window 13 may be in the form of an aperture in the housing 10, which permits a user to view a limited portion of a number sleeve 70 that is configured to move when the dosage knob 12 is turned, to provide a visual indication of a currently programmed dose. The dosage knob 12 is rotated on a helical path with respect to the housing 10 when turned during programming.

In this example, the dosage knob 12 includes one or more formations 71a, 71b, 71c to facilitate attachment of a supplemental device to be described herein below.

The injection device 1 may be configured so that turning the dosage knob 12 causes a mechanical click sound to provide acoustical feedback to a user. The number sleeve 70 mechanically interacts with a piston in insulin container 14. When needle 15 is stuck into a skin portion of a patient, and then injection button 11 is pushed, the insulin dose displayed in display window 13 will be ejected from injection device 1. When the needle 15 of injection device 1 remains for a certain time in the skin portion after the injection button 11 is pushed, a high percentage of the dose is actually injected into the patient's body. Ejection of the insulin dose may also cause a mechanical click sound, which is however different from the sounds produced when using dosage knob 12.

In this embodiment, during delivery of the insulin dose, the dosage knob 12 is turned to its initial position in an axial movement, that is to say without rotation, while the number sleeve 70 is rotated to return to its initial position, e.g. to display a dose of zero units.

Injection device 1 may be used for several injection processes until either the insulin container 14 is empty or the expiration date of the medicament in the injection device 1 (e.g. 28 days after the first use) is reached.

Furthermore, before using injection device 1 for the first time, it may be necessary to perform a so-called “prime shot” to remove air from insulin container 14 and needle 15, for instance by selecting two units of insulin and pressing injection button 11 while holding injection device 1 with the needle 15 upwards. For simplicity of presentation, in the following, it will be assumed that the ejected amounts substantially correspond to the injected doses, so that, for instance the amount of medicament ejected from the injection device 1 is equal to the dose received by the user. Nevertheless, differences (e.g. losses) between the ejected amounts and the injected doses may need to be taken into account.

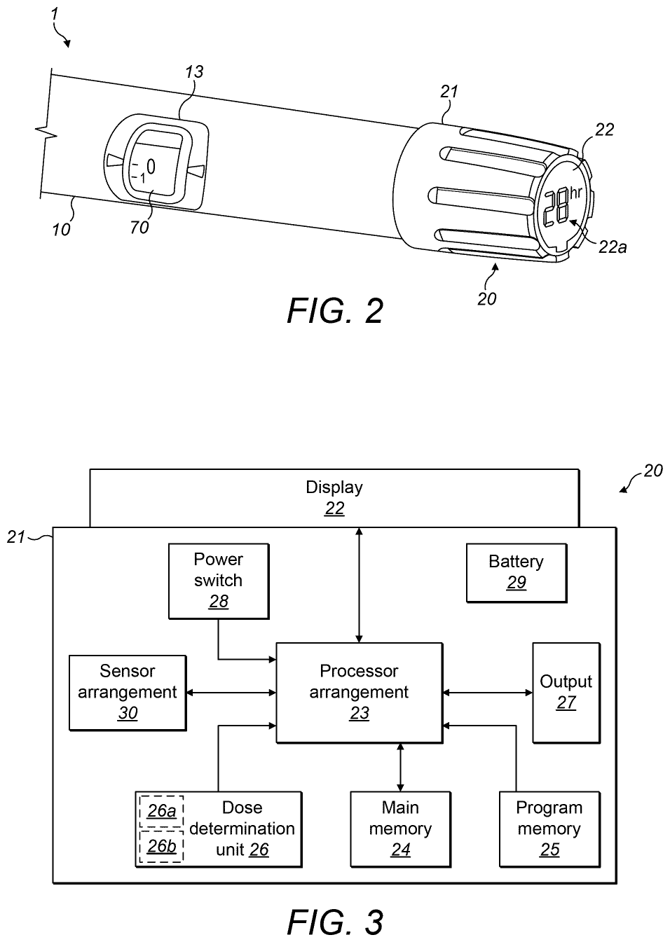

FIG. 2 is a perspective view of one end of the injection device 1 with a supplemental device 20 according to example embodiments attached. In this example, the supplemental device 20 is attached to the injection button 11 of the injection device 1. The supplemental device 20, such as a data collection device, includes a housing 21 and optionally a display 22 for presenting dosage information 22a or other information regarding use of the supplemental device 20 and/or injection device 1 to a user.

The supplemental device 20 has a coupling arrangement (not shown) for coupling the supplemental device 20 to the injection device 1. The coupling arrangement may comprise any suitable coupling means such as one or more clips, latches, ribs, bumps, screw threads, magnets, adhesives or the like. The coupling arrangement interacts with a corresponding coupling feature (not shown) of the injection device 1 to couple the supplemental device 20 and injection device 1 together.

The supplemental device 20 may also have an alignment arrangement (not shown) that interacts with a corresponding alignment feature of the injection device 1 when the supplemental device 20 is coupled to the injection device 1. The alignment arrangement and alignment feature may interact to ensure the supplemental device 20 can be attached to the injection device 1 in only a discrete number of orientations relative to the injection device 1. As should become clear, the presence of an alignment arrangement could be useful in some of the embodiments discussed later below where the supplemental device 20 and injection device 1 need to be coupled in a predetermined configuration. The discrete number of orientations is preferably one or two. If the discrete number is one then the supplemental device 20 can be attached to the injection device 1 in only one orientation relative to the injection device 1, thereby ensuring the supplemental device 20 is always coupled to the injection device 1 in the same relative position. If the discrete number is two then the supplemental device 20 can be attached to the injection device 1 in only two different orientations relative to the injection device 1, thereby ensuring the supplemental device 20 is always coupled to the injection device 1 in one of the two possible orientations. If the discrete number is two then this may mean the alignment arrangement and supplemental device 20 are arranged to have a rotational symmetry of order two, such that the two possible orientations are separated by a 180 degrees rotation of the supplemental device 20 about an axis.

The discrete number of orientations may be three or four, or a number higher than four. If the discrete number is three then the supplemental device 20 can be attached to the injection device 1 in only three different orientations relative to the injection device 1. The alignment arrangement and supplemental device 20 may be arranged to have a rotational symmetry of order three, such that the three possible orientations are separated by a 120 degrees rotation of the supplemental device 20 about an axis. If the discrete number is four then the supplemental device 20 can be attached to the injection device 1 in only four different orientations relative to the injection device 1. The alignment arrangement and supplemental device 20 may be arranged to have a rotational symmetry of order four, such that the four possible orientations are separated by a 90 degrees rotation of the supplemental device 20 about an axis.

FIG. 3 is a schematic illustration of various components that may be included in the supplemental device 20. As shown in FIG. 3, the supplemental device 20 includes a processor arrangement 23 including one or more processors, such as a microprocessor, a Digital Signal Processor (DSP), Application Specific Integrated Circuit (ASIC), Field Programmable Gate Array (FPGA) or the like, together with memory units 24, 25, including program memory 24 and main memory 25, which can store software for execution by the processor arrangement 23.

A communication interface 27 may be provided, which may be a wireless communications interface for communicating with another device via a wireless network such as wi-fi or Bluetooth®, or an interface for a wired communications link, such as a socket for receiving a Universal Series Bus (USB), mini-USB or micro-USB connector. The other device may be a mobile computing device such as a smartphone.

A power switch 28 is provided, together with a battery 29. In one example, the power switch 28 is configured to respond to pressure applied to the display 22 by powering the supplemental device 20 on or off. The battery 29 provides power to one or more components of the supplemental device 20. In other examples, power can be provided from a source other than a battery 29, such as using wireless power transfer, a solar cell, or a wired connection to a power supply external to the supplemental device 20. In some examples, the power switch 28 may be omitted.

A sensor arrangement 30 is provided, for sensing an identification feature of the injection device 1. The identification feature is a feature of the injection device 1 that indicates the type of the injection device 1. The identification feature can be sensed by the sensor arrangement 30 to determine the type of the injection device 1. By sensing the identification feature, the sensor arrangement 30 is used to determine a property associated with that feature, which property corresponds to the type of the injection device 1. The sensor arrangement 30 senses the identification feature when the supplemental device 20 is coupled to the injection device 1.

The type of the injection device 1 may relate to the type of medicament contained in the injection device 1. For example, the type may indicate the name of the medicament contained within the injection device or the concentration of the medicament. In some examples, the type of the injection device 1 may relate to a type of dose dialling mechanism present in the injection device 1, or a type of dispensing mechanism used in the injection device 1. If the type of the injection device 1 relates to a type of dose dialling mechanism present in the injection device 1 or a type of dispensing mechanism used in the injection device 1 then the determined type of the injection device 1 may be used to calibrate a dose determination unit 26.

The type of the injection device 1 may provide a unique identification of an injection device 1, such as a serial number, or there may be more than one injection device 1 having the same type.

The sensor arrangement 30 can take a number of forms, as exemplified in the example embodiments discussed below. In each example, the processor arrangement 23 may be configured to cause the supplemental device 20 to use the sensor arrangement 30 to sense an identification feature of the injection device 1, and determine a type of the injection device 1 based on the sensed identification feature, such that it can differentiate between different types of injection device 1 and modify its operation accordingly.

In any of the embodiments discussed below, a dose determination unit 26 comprising one or more sensors may be provided in the supplemental device 20. The dose determination unit 26 is used by the processor arrangement 23 to determine a dose. For example, the dose determination unit 26 may be used by the processor arrangement 23 to determine a dose dialled into the injection device 1 and/or a dose dispensed by the injection device 1. In the particular example of FIG. 3, the dose determination unit 26 comprises an optical encoder, including a light source 26a, such as a light emitting diode (LED) and a light detector 26b, such as an optical transducer. However, the dose determination unit 26 may alternatively or additionally comprise other suitable means for dose determination such as a Hall Effect sensor, rotary encoder or the like.

In some examples, the dose may be determined by the processor arrangement 23 based on the type of injection device 1 determined using the sensor arrangement 30. In other words, the processor arrangement 23 may determine the dose using the both the dose determination unit 26 and the determined type of injection device 1. For example, the type of the injection device 1 may indicate the concentration of the medicament contained in the injection device 1. The processor arrangement 23 may therefore use the concentration to determine how many units of medicament were dispensed. In another example, the type may indicate the number of units dispensed per incremental movement of a dose delivery mechanism contained in the injection device 1. The processor arrangement 23 may therefore use this factor in combination with a number of incremental movements detected by the dose determination unit 26 to calculate a dose. The supplemental device 20 may therefore be adaptable for use with a variety of different types of injection device 1.

Having determined the type of the injection device 1 to which the supplemental device 20 is coupled, the processor arrangement 23 may be configured to transmit data indicating the type of the injection device 1 to an external computing device such as a smartphone, via the communication interface 27. Additionally or alternatively, the processor arrangement 23 may be configured to transmit the dose determined using the dose determination unit 26 to the external computing device.

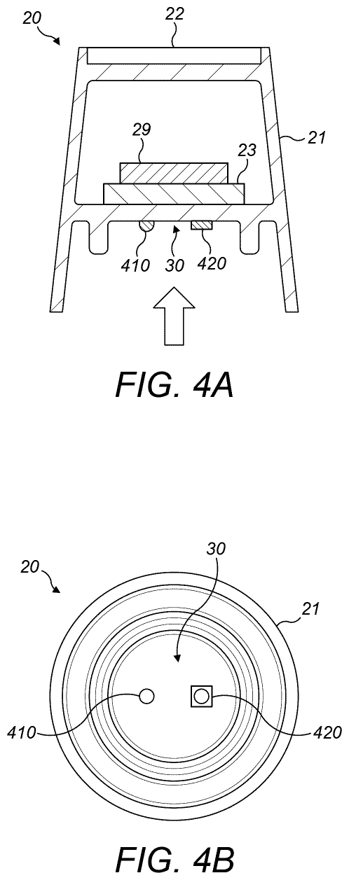

FIG. 4A shows a cross-sectional side view of a supplemental device 20 according to a first embodiment of the present disclosure. The supplemental device 20 generally has the same structure as the supplemental device 20 shown in FIG. 2. In this embodiment, the sensor arrangement 30 comprises a light source 410, such as an LED, and a color sensor 420. The light source 410 is arranged on the supplemental device 20 so that it illuminates a portion of the injection device 1 when the supplemental device 20 is coupled to the injection device 1. The color sensor 420 is arranged so that it can detect light reflected from the illuminated portion of the injection device 1 when coupled to the injection device 1, and thereby detect the color of the portion of the injection device 1.

In this embodiment, the identification feature of the injection device 1 is the colored portion of the injection device 1 detected by the color sensor 420. The colored portion 1 could be a portion of the injection button 11 of the injection device 1, to which the supplemental device 20 is coupled.

FIG. 4B shows the supplemental device 20 of FIG. 4A when viewed from below, in the direction of the large arrow shown in FIG. 4A.

The color of the portion of the injection device 1 indicates the type of injection device 1. This provides a simple means of identifying the type of an injection device 1. Only a small change to the manufacturing process of the injection device 1 may be required. The portion of the injection device 1 may be formed from a material having the desired color, or the color may be applied to the portion of the injection device 1 after manufacture of the portion of the injection device 1, for example by printing or spraying the color onto the portion.

The processor arrangement 23 of the supplemental device 20 controls the light source 410 to illuminate the portion of the injection device 1. The color sensor 420 receives reflected light from the illuminated portion of the injection device 1 and outputs a signal corresponding to the light received from the illuminated portion, and therefore the color of the portion. The signal may be received by the processor arrangement 23, which determines a color based on the signal and therefore determines the type of injection device 1, for example by comparing the identified color with a lookup table stored in the memory 24 of the supplemental device 20. The lookup table can contain a list of colors and the corresponding type of injection device that each color is indicative of.

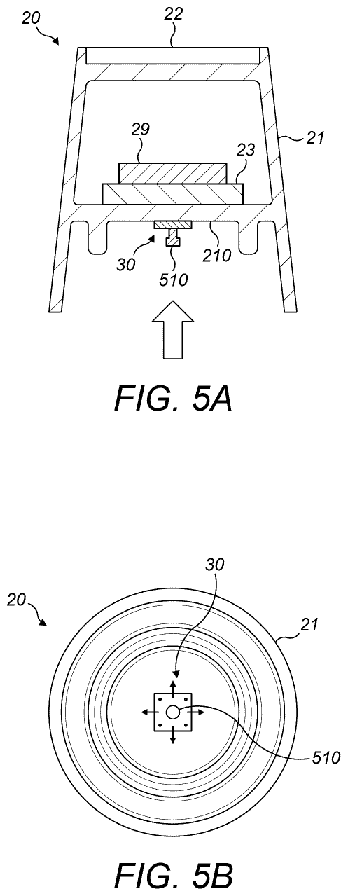

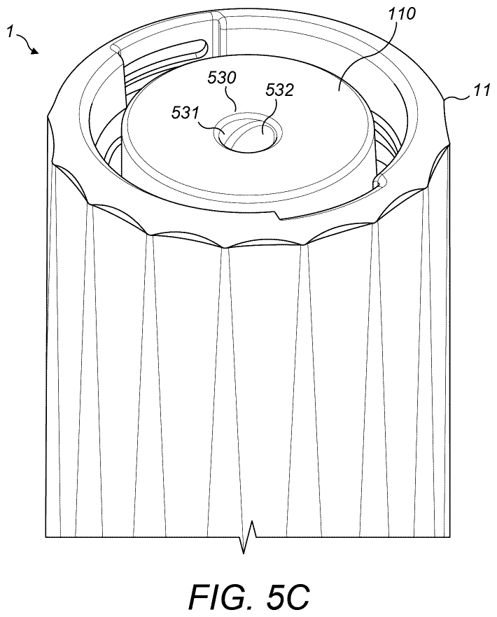

In a second embodiment as illustrated in FIG. 5A, the sensor arrangement 30 comprises one or more switches to be activated by the injection device 1, wherein the activation of the switches is used to determine the type of the injection device 1. FIG. 5A shows the sensor arrangement 30 comprising a navigation switch 510, sometimes known as a joystick switch. The navigation switch 510 is located on the lower surface 210 of the supplemental device 20 so that it faces the injection device 1 when the supplemental device 20 is coupled to the injection device 1.

FIG. 5B shows the supplemental device 20 of FIG. 5A when viewed from below, in the direction of the large arrow in FIG. 5A.

FIG. 5B shows that the navigation switch 510 can be toggled between four positions, as indicated by the four small arrows in FIG. 5B. However, in other examples the navigation switch 510 may be able to toggle between a different number of positions such as two positions, three positions, five positions or more. Each position of the navigation switch 510 results in a different signal being sent to the processor arrangement 23 by the navigation switch 510. Furthermore, each position, and therefore signal sent to the processor arrangement 23, corresponds to a particular type of injection device 1. The processor arrangement 23 is able to determine which position the navigation switch 510 is in based on the signal sent from the navigation switch 510 and therefore determine the type of the injection device 1.

The navigation switch 510 may be in a neutral position before the supplemental device 20 is attached to the injection device 10, as shown in FIG. 5B.

FIG. 5C shows a portion of the injection device 1 to which the supplemental device 20 is to be attached, in this case the injection button 11 of the injection device 1. As the supplemental device 20 is coupled to the injection device 1, a biasing feature 530 on the injection device 1 biases the navigation switch 510 into one of the positions of the navigation switch 510. In this embodiment, the identification feature will be the biasing feature 530.

A different biasing feature 530 can be used for each different type of injection device 1, with each biasing feature 530 causing the navigation switch 510 to be moved into a different position. The position that the navigation switch 510 is moved into is therefore indicative of the type of the injection device 1 and can be detected by the processor arrangement 23.

FIG. 5C shows the biasing feature 530 being located on an upper surface 110 of the injection button 11. The biasing feature 530 comprises a recess 531 formed in the upper surface 110 of the injection button 11. The recess 531 has a sloping lower surface 532. The sloping lower surface 532 of the recess 531 makes contact with the navigation switch 510 as the supplemental device 20 is brought towards the injection device 1 during attachment. The navigation switch 510 is deflected into a position by a force exerted by the sloping lower surface 532. The position the navigation switch 510 is forced into will depend on the direction of the slope relative to the navigation switch 510, and will be detected by the processor arrangement 23.

The determined position of the navigation switch 510 may be used by the processor arrangement 23 to determine the type of injection device 1, for example by comparing the position with a lookup table stored in the memory 24 of the supplemental device 20. The lookup table can contain a list of positions of the navigation switch 510 and the corresponding types of injection device that they are indicative of.

As discussed previously, the supplemental device 20 may have an alignment arrangement and the injection device 1 have a corresponding alignment feature that ensures the supplemental device 20 and injection device 1 can only be coupled in one alignment. This means the same injection device 1 will always move the navigation switch 510 into the same position. The alignment arrangement and/or feature may comprise one or more grooves, ribs, bumps, clips, recesses, keys or the like.

In some examples, the sensor arrangement 30 may comprise more than one switch that can be activated upon attachment of the supplemental device 20 to the injection device 1. One or more of the switches may be a navigation switch 510 as discussed above. In other examples, the sensor arrangement 30 may comprise a plurality of push switches such as microswitches. The push switches may be arranged in the supplemental device 20 so that they interact with corresponding biasing features on the injection device 1, such as biasing features on the injection button 11.

Each push switch may be pressed or released depending on the presence of a corresponding biasing feature on the injection device 1. In some examples a biasing feature may be a protrusion arranged to press a corresponding push switch when the supplemental device 20 is coupled to the injection device 1. In other examples the biasing feature may be a recess that prevents a corresponding push switch from being pressed when the supplemental device 20 is coupled to the injection device 1.

The processor arrangement 23 is configured to determine which push switches have been pressed by the injection device 1. The particular combination of pushed switches detected by the processor arrangement 23 may be used by the processor arrangement 23 to determine the type of injection device 1, for example by comparing the particular combination of pushed switches with a lookup table stored in the memory 24 of the supplemental device 20. The lookup table can contain a list of different combinations of pushed switches and the corresponding types of injection device 1 that they are indicative of.

A single push switch gives one bit of information, so n switches will provide a resolution of 2n different types of device.

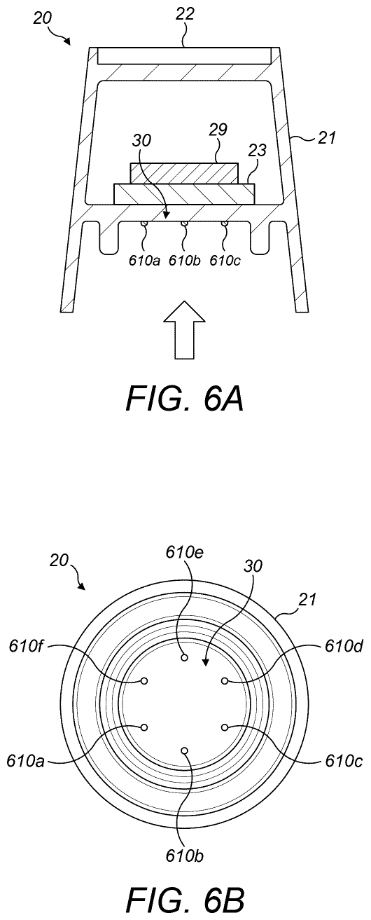

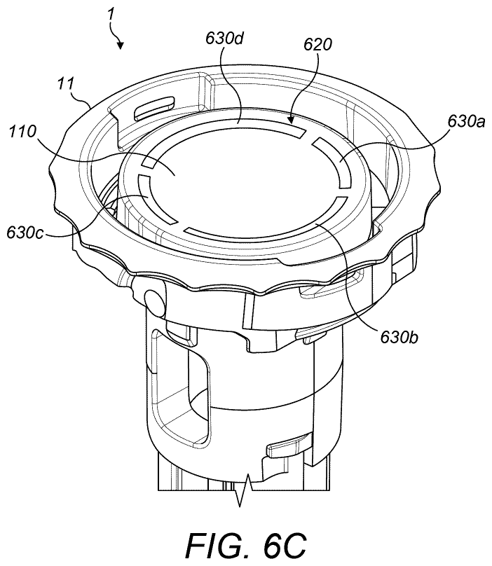

According to a third embodiment, as shown in FIGS. 6A and 6B, the sensor arrangement 30 may comprise a plurality of electrical contacts 610a-f. The contacts 610a-f are arranged to make electrical contact with a conductive track 620 formed on the injection device 1 when the supplemental device 20 is coupled to the injection device 1. In this particular embodiment, the identification feature is the conductive track 620 on the injection device 1.

FIG. 6B shows the supplemental device 20 of FIG. 6A when viewed from below, in the direction of the large arrow in FIG. 6A. It can be seen that the contacts 610a-f are arranged in a circular pattern. This provides a particularly compact arrangement, however the contacts 610a-f are not limited to such an arrangement.

FIG. 6C shows a portion of the injection device 1 to which the supplemental device 20 is to be attached, in this case the injection button 11. The conductive track 620 is formed on a surface of the injection device 1, in this case on an upper surface 110 of the injection button 11. The conductive track 620 is formed of an electrically conductive material and may be provided on the injection device 1 by printing, spraying, transferring, or any other suitable application means.

It can be seen in FIG. 6C that the conductive track 620 forms a circular shape that corresponds with the circular arrangement of the contacts 610a-f, however the conductive track 620 is not limited to such a circular shape. The conductive track 620 comprises one or more conductive track segments 630a-d, separated by non-conductive gaps. FIG. 6C shows the conductive track 620 comprising four segments 630a-d, however this is by way of example only, and any number of segments 630a-d may be used.

When the supplemental device 20 is coupled to the injection device 1, each segment 630a-d electrically couples two or more of the contacts 610a-f together. Each type of injection device 1 may have a unique arrangement of the track 620 and track segments 630a-d. The processor arrangement 23 is configured to distinguish between the different types of injection device 1 by detecting the particular arrangement of the track 620 using the electrical contacts 610a-f.

The processor arrangement 23 is configured to poll each of the contacts 610a-f by applying a current to each contact 610a-f. When a particular contact 610a-f is being polled, the processor arrangement 23 at the same time can detect whether current is flowing through any of the other contacts 610a-f, via a segment 630a-d of the track 620. This will happen when the contact 610a-f being polled is electrically connected to another one of the contacts 610a-f via a segment 630a-d of the conductive track 620.

If a current is able to flow between two contacts 610a-f via a segment of conductive track 620 then this may be considered by the processor arrangement 23 to be a binary signal of ‘1’. However if no current is able to flow between the two contacts 610a-f, because there is no segment 630a-d of conductive track 620 joining the two contacts 610a-f, then this may be considered a binary signal of ‘0’. The processor arrangement 23 may identify a binary code in the segments 630a-d of conductive track 620 by polling each of the contacts 610a-f. The binary code may then be used by the processor arrangement 23 to determine the type of injection device 1, for example by comparing the binary code to a lookup table stored in the memory 24, where each binary code corresponds to a type of injection device 1.

FIG. 6A and FIG. 6B both show six contacts 610a-f being used, however any number of contacts 610a-f from two upwards could be used instead. For n contacts 610a-f, 2n different types of injection device 1 could be resolved by the processor arrangement 23.

As discussed previously, the supplemental device 20 may have an alignment arrangement and the injection device 1 have a corresponding alignment feature that ensures the supplemental device 20 and injection device 1 can only be coupled in one relative orientation. This will ensure the contacts 610a-f and the segments 630a-d of conductive track 620 are correctly aligned each time the supplemental device 20 and injection device 1 are coupled. In other examples, the supplemental device 20 may have an alignment arrangement and the injection device 1 have a corresponding alignment feature that ensures the supplemental device 20 can only be coupled to the injection device 1 in two different orientations relative to the injection device 1. The contacts 610a-f and the segments 630a-d of conductive track 620 may be arranged so that the supplemental device 20 is able to determine the type of injection device 1 when it is coupled to the injection device 1 in both the first orientation and the second orientation. If the supplemental device 20 can only be coupled to the injection device 1 in a different number of orientations such as three, four or more then the contacts 610a-f and the segments 630a-d of conductive track 620 may be arranged so that the supplemental device 20 is able to determine the type of injection device 1 when it is coupled to the injection device 1 in each of the discrete orientations.

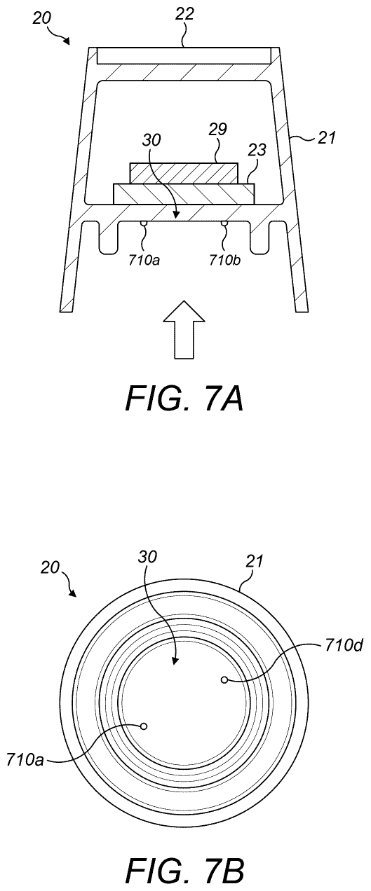

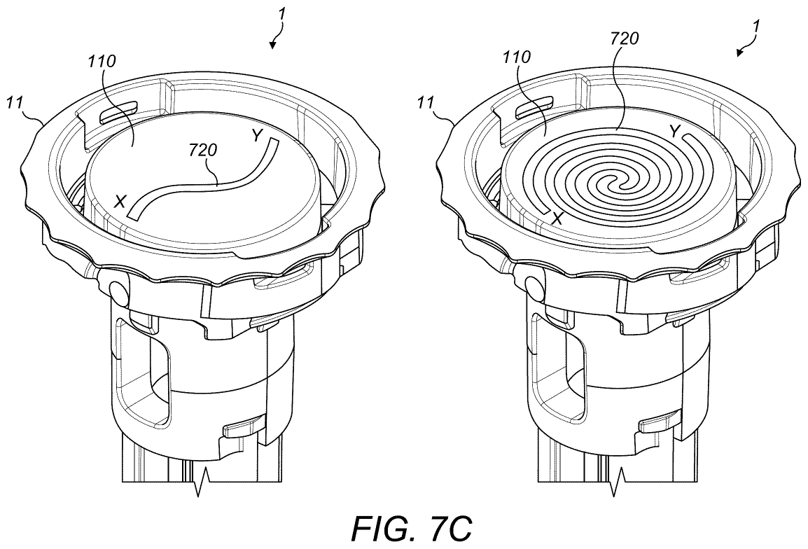

FIG. 7A shows a fourth embodiment which is similar to the embodiment shown in FIG. 6A, with the sensor arrangement 30 comprising a pair of electrical contacts 710a-b, each contact 710a-b arranged to protrude from the supplemental device 20. FIG. 7B shows the supplemental device 20 of FIG. 7A when viewed from below, in the direction of the large arrow in FIG. 7A.

Each contact 710a-b is arranged to make electrical contact with a different point on an electrically conductive track 720 formed on a surface of an injection device 1. In this embodiment, the identification feature is the conductive track 720. FIG. 7C shows portions of two different injection devices 1 to which the supplemental device 20 can be attached. In both cases the portion is an injection button 11. The conductive track 720 is formed on a surface of the injection device 1, in this case on an upper surface 110 of the injection button 11. The conductive track 720 on the left injection button 11 in FIG. 7C is shorter than the conductive track on the right injection button 11 in FIG. 7C.

The conductive track 720 is formed of an electrically conductive material and may be provided on the injection device 1 by printing, spraying, transferring, or any other suitable application means.

When the supplemental device 20 is coupled to the injection device 1, the contacts 710a-b make electrical contact with two different points on the conductive track 720. In this example, contact 710a of FIG. 7A will make contact with a point on the track 720 of FIG. 7C near the label X, while contact 710b will make contact with a point on the track 720 of FIG. 7C near the label Y. These two points X and Y may be at different ends of the conductive track 720. The resistance of the conductive track 720 between the two points X and Y is measured by the supplemental device 20 passing a current between the electrical contacts 710a-b via the conductive track 720. Measurement of the electrical resistance may be carried out under the control of the processor arrangement 23.

The measured electrical resistance may correspond to the type of injection device 1. Different conductive tracks 720 having different resistances may be applied to different injection devices 1. Each type of injection device may have a conductive track 720 having a unique resistance which can be detected by the supplemental device 20 and used to determine the injection device 1 type. The processor arrangement 23 may measure the resistance of the conductive track 720 using the contacts 710a-b and then determine the type of injection device 1 based on the measured resistance. For example, the processor arrangement 23 may compare the measured resistance to values stored in a lookup table in the memory 24 or elsewhere. The lookup table may contain resistance values and the associated types of injection device 1. The lookup table may contain resistance ranges rather than specific values to compensate for slight variations in the measured resistance.

As mentioned previously, the conductive track 720 shown on the injection device 1 on the left of FIG. 7C is shorter than the conductive track 720 on the injection device 1 shown on the right of FIG. 7C. The conductive track 720 having the longer length will generally have the higher resistance, assuming the conductive tracks 720 have identical widths and thicknesses, and are made of the same material. Changing the length of the conductive track 720 provided on the injection device 1 therefore provides a simple means of varying the resistance of the track 720 and therefore differentiating between types of injection device 1. Alternatively or in addition, the resistance of the conductive track 720 could be changed by varying one or more of the thickness of the track 720, the width of the track 720, or the material the track 720 is formed from.

The supplemental device 20 of FIG. 7A will be able to distinguish between the two injection devices shown in FIG. 7C based on the resistance of the respective track 720 formed on each injection device 1.

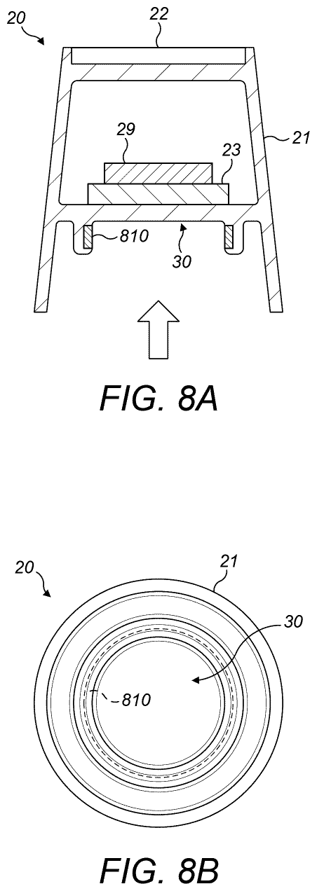

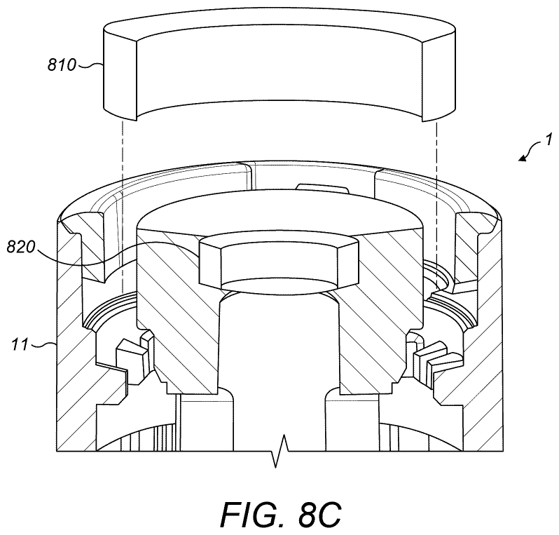

In a fifth embodiment as shown in FIG. 8A, the sensor arrangement 30 may comprise a magnetic field sensor. The magnetic field sensor may be arranged to detect a magnetic field generated by the injection device 1 when the supplemental device 20 is coupled to the injection device 1. The magnetic field generated by the injection device 1 may be a permanent magnetic field generated by a permanent magnet located in the injection device 1, or it may be a temporary magnetic field generated by an electromagnetic interaction between the supplemental device 20 and the injection device 1. The processor arrangement 23 may be configured to measure or detect the magnetic field using the magnetic field sensor, the resulting measurement being compared with values stored in a lookup table to determine the type of the injection device 1.

FIG. 8A shows a particular example in which the sensor arrangement 30 comprises an inductor such as a coil 810 acting as a magnetic field sensor. The injection device 1 also contains an inductor, such as a coil 820, as shown in FIG. 8C. In this embodiment, the identification feature is the coil 820 in the injection device 1.

FIG. 8B shows the supplemental device 20 of FIG. 8A when viewed from below, in the direction of the large arrow in FIG. 8A. As can be seen in FIG. 8A and FIG. 8B, the coil 810 of the supplemental device is a circular coil that extends from a lower surface of the supplemental device 20.

FIG. 8C also shows the coil 810 of the supplemental device 20, to illustrate how it surrounds the coil 820 of the injection device 1 when the supplemental device 20 and injection device 1 are coupled. The dotted lines indicate how the coil 810 of the supplemental device 20 is accommodated in the injection button 11 of the injection device 1 when the supplemental device 20 is coupled to the injection device 1. The coil 810 of the supplemental device 20 is accommodated such that the coil 810 of the supplemental device 20 and the coil 820 of the injection device 1 are concentric.

The coil 810 in the supplemental device 20 and the coil 820 in the injection device 1 are arranged so that they are electromagnetically coupled when the supplemental device 20 is attached to the injection device 1. The coil 810 in the supplemental device 20 has a known, fixed inductance. The coil 820 in the injection device 1 has an inductance that will vary between injection devices 1, in dependence on the type of injection device 1. That is, each type of injection device may have a coil 820 with a unique inductance so that the unique inductance corresponds to the injection device 1 type.

The processor arrangement 23 may apply a current to the coil 810 in the supplemental device 20 causing a magnetic field to be generated, which interacts with the coil 820 in the injection device 1 through mutual inductance. The processor arrangement 23 may be configured to measure the total inductance of the coupled coils 810, 820. This measurement may then be used to determine the type of the injection device 1, for example by comparing the inductance value to values in a lookup table, for example stored in the memory 24. Different inductance values—or ranges of values—will correspond to different types of injection device 1. The inductance can therefore be used to identify the type of the injection device 1.

In some examples, the magnetic field sensor comprises one or more Hall effect sensors arranged to detect one or more magnets in the injection device 1 when the supplemental device 20 is coupled to the injection device 1. For different types of injection device 1, the one or more magnets will be configured so that they induce different responses in the one or more Hall effect sensors. The processor arrangement 23 is therefore able to distinguish between different types of injection device 1 depending on the detected response output by the one or more Hall effect sensors.

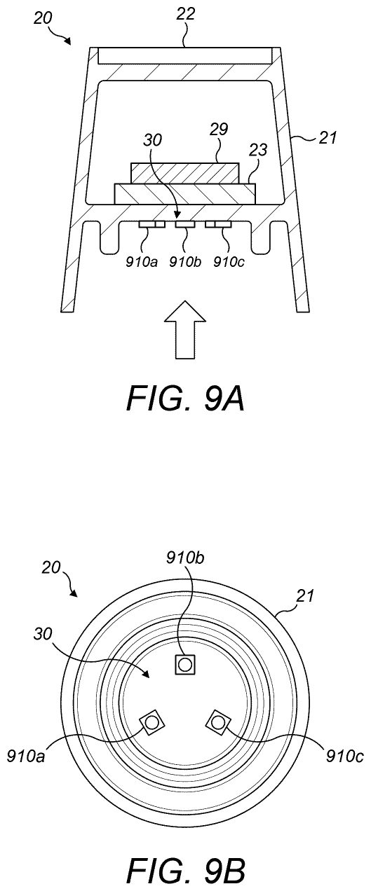

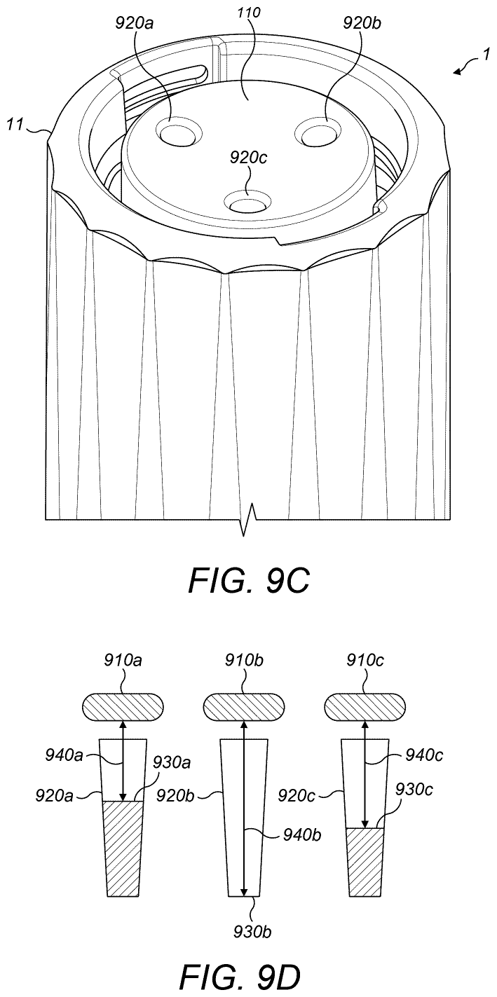

FIG. 9A shows sixth embodiment in which the sensor arrangement 30 comprises three proximity sensors 910a-c directed towards the injection device 1 when the supplemental device 20 is coupled to the injection device 1. In this example, each proximity sensor 910a-c is an infrared transceiver. FIG. 9B shows the supplemental device 20 of FIG. 9A when viewed from below, in the direction of the arrow in FIG. 9A.

The proximity sensors 910a-c are used to determine a distance between the supplemental device 20 and one or more corresponding features of the injection device 1. The distance between the supplemental device 20 and one or more corresponding features of the injection device 1 may correspond to the type of the injection device 1. In this embodiment, the identification feature is the one or more corresponding features of the injection device 1 that are used by the one or more proximity sensors 910a-c to determine the distance.

FIG. 9C shows a portion of the injection device 1 to which the supplemental device 20 is to be attached, in this case the injection button 11. Three holes 920a-c are formed on a surface of the injection device 1, in this case on an upper surface 110 of the injection button 11. Each hole 920a-c corresponds to one of the proximity sensors 910a-c. The depth of each hole 920a-c may vary, as illustrated in schematic FIG. 9D.

FIG. 9D shows each hole 920a-c having a corresponding bottom surface 930a-c. Each proximity sensor 910a-c is positioned above a corresponding hole 920a-c such that the proximity sensor 910a-c may determine a distance 940a-c between itself and the corresponding bottom surface 930a-c of a respective hole 920a-c.

Each proximity sensor 910a-c may determine a value of a corresponding distance 940a-c, or it may simply determine whether or not the corresponding distance 940a-c meets or exceeds a threshold distance. In the case that the proximity sensor 910a-c determines whether or not the corresponding distance 940a-c meets or exceeds a threshold distance, the proximity sensor 910a-c can effectively be used as a binary switch. However, if the proximity sensor 910a-c is able to distinguish between three or more distances 940a-c then each distinct distance can be indicative of a different type of injection device 1.

Different types of injection device 1 can be differentiated by adjusting the depths/heights of the various features sensed by the proximity sensors 910a-c, thereby adjusting the distances between the features and the corresponding proximity sensors 910a-c when the supplemental device 20 is coupled to the injection device 1. For example, the depths 940a-c of the three holes 920a-c may vary depending on the type of injection device. The processor arrangement 23 will be able to distinguish between different combinations of outputs of the proximity sensors 910a-c. Each combination of outputs corresponds to a type of injection device 1. The processor arrangement 23 may compare the particular combination of outputs to values in a lookup table stored in the memory 24, to determine the corresponding type of injection device 1.

While FIG. 9A shows three proximity sensors 910a-c, the present disclosure is not limited to such number and there may be more or fewer proximity sensors 910a-c than this.

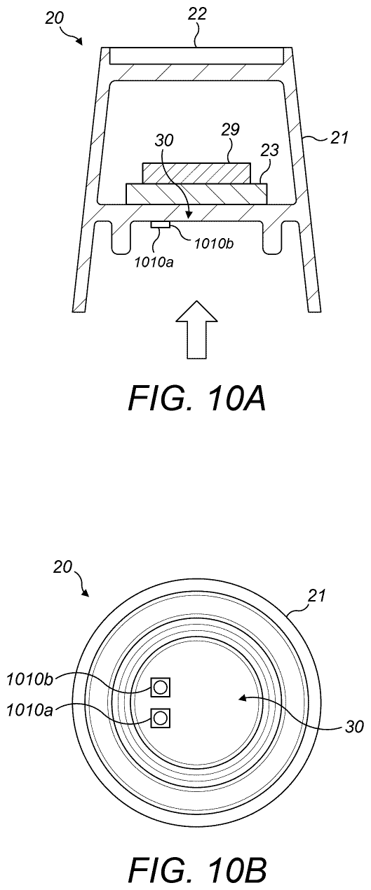

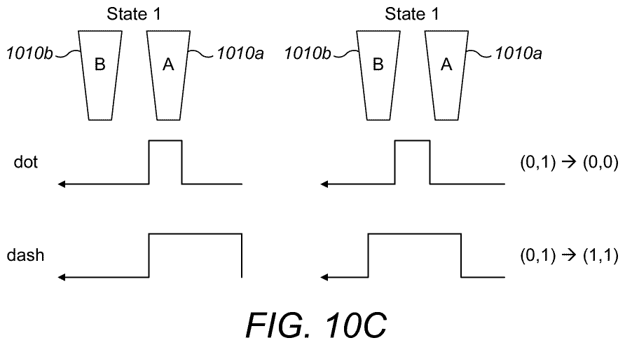

FIG. 10A shows a seventh embodiment in which the sensor arrangement 30 comprises a pair of infrared sensors 1010a-b. Infrared sensor 1010b is obscured behind infrared sensor 1010a in FIG. 10A, but can be seen in FIG. 10B. FIG. 10B shows the supplemental device 20 of FIG. 10A from below, in the direction of the arrow shown in FIG. 10A. The infrared sensors 1010a-b are arranged to read an encoded track on the injection device 1, the identification feature therefore being the encoded track formed on the injection device 1.

The encoded track may appear similar to the conductive tack 620 discussed previously in relation to FIG. 6C and may be provided on a similar location on the injection device 1, such as the on a surface of the injection button 11. The track consists of two types of segments, one type that induces a first type of response in the infrared sensors 1010a-b and another type that induces a second type of response in the infrared sensors 1010a-b. The track may be formed from grooves in a surface of the injection device, or by a material printed on the surface. Where the track is formed from grooves, sections of the surface having a groove formed in it can be considered the first type of segment that induces the first type of response, while sections of the surface without a groove can be considered the second type of segment that induces the second type of response. Where the track is formed from a material printed on the surface, sections having a relatively high infrared reflectivity may be considered the first type of segment, while segments having a relatively low infrared reflectivity may be considered the second type of segment.

The segments can have two different lengths, a shorter length representing a ‘dot’ and a longer length representing a ‘dash’, as illustrated in FIG. 10C. The segments can therefore be used to provide a binary code in the encoded track.

The infrared sensors 1010a-b are arranged to read the encoded track at proximate, but different locations along the track. The infrared sensors 1010a-b and encoded track are arranged such that both of the infrared sensors 1010a-b scan along the length of the track as the supplemental device 20 is coupled to the injection device 1. In other words, the two infrared sensors 1010a-b are both arranged to read the encoded track as the supplemental device 20 is moved relative to the injection device 1 during coupling of the supplemental device 20 to the injection device 1. The first infrared sensor 1010a reads the track ahead of the second infrared sensor 1010b when coupling the supplemental device 20 to the injection device 1.

In the particular embodiment shown in FIG. 10A and FIG. 10B, the supplemental device 20 is configured to be attached to the injection device 1 using a bayonet-style coupling, which involves rotation of the supplemental device 20 relative to the injection device 1. At the start of coupling, the pair of infrared sensors 1010a-b may be directed at different but proximate points of the track. The track would be circular, similar to the track 620 shown in FIG. 6C. As the supplemental device 20 and injection device 1 are coupled by rotating the supplemental device 20 relative to the injection device 1, the pair of infrared sensors 1010a-b rotate relative to injection device 1 so that they both move along the length of the track, reading the binary code as they move.

The infrared sensors 1010a-b and track segments are arranged so that only one infrared sensor 1010a-b at a time is able to read a short ‘dot’ segment, whereas both infrared sensors 1010a-b are able to read a longer ‘dash’ segment of track at the same time, as illustrated in FIG. 10C. As a result, the two infrared sensors 1010a-b are able to read binary information independent of the speed at which the supplemental device 20 and injection device 1 are coupled.

The processor arrangement 23 can use the binary code read from the encoded track to determine the type of injection device 1, for example by comparing the binary code to values in a lookup table. The lookup table may list different binary codes and the corresponding types of injection device 1 they each represent.

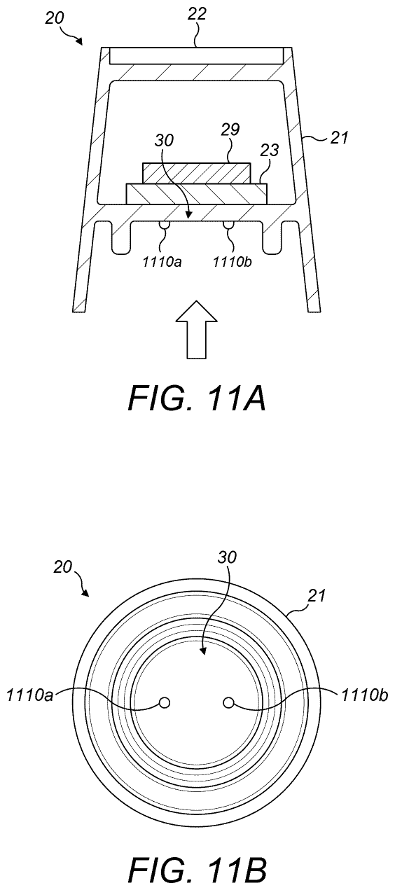

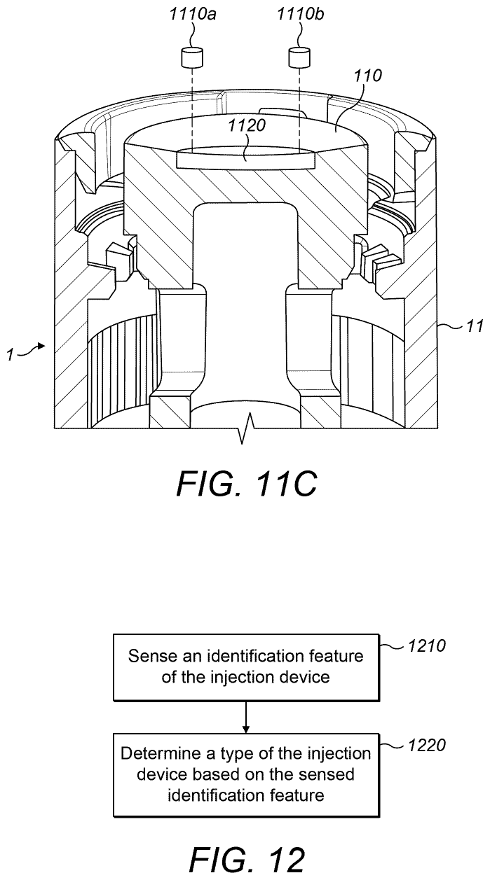

According to an eighth embodiment as illustrated in FIG. 11A, the sensor arrangement 30 comprises pins 1110a-b configured to make electrical contact with a non-volatile memory 1120 arranged in the injection device 1, when the supplemental device 20 is coupled to the injection device 1. In this embodiment, the identification feature is the non-volatile memory 1120.

FIG. 11B shows the supplemental device of FIG. 11A when viewed from below, in the directions indicated by the arrow in FIG. 11A.

FIG. 11C shows a portion of the injection device 1 to which the supplemental device 20 is to be attached, in this case the injection button 11. The non-volatile memory 1120 is located in the injection device 1, in this case in the injection button 11. The non-volatile memory 1120 may be an electrically erasable programmable read-only memory (EEPROM), for example. The non-volatile memory 1120 stores data that can be used to identify the type of injection device. The data could include one or more serial numbers, batch dates, drug origins or the like.

When the supplemental device 20 is coupled to the injection device 1, the pins 1110a-b make electrical contact with the non-volatile memory 1120. In some examples, the pins 1110a-b make direct electrical contact with corresponding pins of the non-volatile memory, for example the corresponding pins of an EEPROM chip. In such an example, power may be supplied to non-volatile memory 1120 from the supplemental device 20 via the pins 1110a-b so that separate power supply for the EEPROM needs to be provided in the injection device 1. The non-volatile memory 1120 may be located at the upper surface 110 of the injection button 11 in such an example. In other examples, the pins 1110a-b of the supplemental device 20 make electrical contact with the non-volatile memory 1120 via one or more electrical connectors (not shown) provided in the injection device 1, that provide an electrical connection between the pins 1110a-b and the pins of the non-volatile memory 1120.

The processor arrangement 23 of the supplemental device 20 can read the data from the non-volatile memory 1120 via the pins 1110a-b and determine the type of the injection device 1 based on the data.

FIGS. 11A-C show two pins 1110a-b, however any number of pins may be used. In particular, there may be one pin 1110 provided for each corresponding pin of the non-volatile memory 1120.

Aspects of the present disclosure also relate to a system comprising any of the supplemental devices 20 discussed herein, and a corresponding injection device 1 comprising an identification feature, wherein the processor arrangement 23 is configured to cause the supplemental device 20 to use the sensor arrangement 30 to sense the identification feature of the injection device 1, and determine a type of the injection device 1 based on the sensed identification feature.

Aspects of the present disclosure also relate to a method of using an aforementioned supplemental device 20 and a corresponding injection device 1. FIG. 12 is a flowchart illustrating a method of identifying a type of injection device 1 using a supplemental device 20 coupled to the injection device 1, where the supplemental device 20 comprises a sensor arrangement 30 and a processor arrangement 23.

In step 1210, the processor arrangement 23 causes the sensor arrangement 30 to sense an identification feature of the injection device 1. Sensing the identification feature may comprise the sensor arrangement 30 interacting with the identification feature and outputting a signal corresponding to the identification feature. The signal may represent a value associated with the identification feature.

In step 1220, the processor arrangement 23 then determines a type of the injection device 1 based on the sensed identification feature, such that it can differentiate between different types of injection device and modify its operation accordingly. In particular, the processor arrangement 23 may determine the type of the injection device 1 based on the signal output by the sensor arrangement 30. The processor arrangement 23 may compare the signal, or a value derived from the signal, to a plurality of signals or values stored in a lookup table, where each stored signal or value corresponds to a type of injection device. The processor arrangement 23 may determine the type of the injection device 1 based on a result of the comparison.

Aspects of the present disclosure also relate to an injection device 1 comprising one or more of the identification features disclosed herein, the identification feature indicating a type of the injection device 1, and wherein the injection device 1 is configured to be coupled to a supplemental device 20, the identification feature 1 arranged to be sensed by the supplemental device 20 to determine the type of the injection device 1. For example, aspects of the present disclosure include an injection device 1 comprising a conductive track on a surface of the injection device 1 as discussed in relation to FIG. 7C.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N- tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl- ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten.

An example of an oligonucleotide is, for example: mipomersen sodium (Kynamro®), a cholesterol-reducing antisense therapeutic for the treatment of familial hypercholesterolemia.

Examples of DPP4 inhibitors are Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

1-15. (canceled)

16. A supplemental device configured to be coupled to an injection device, the supplemental device comprising:
a sensor arrangement; and
a processor configured to cause the supplemental device to:
use the sensor arrangement to sense an identification feature of the injection device, and
determine a type of the injection device based on the sensed identification feature.

17. The supplemental device according to claim 16, further comprising:
a dose determination unit,
wherein the processor is further configured to cause the supplemental device to determine a dose of medicament contained in the injection device by using the dose determination unit, wherein the dose is determined based on the determined type of the injection device.

18. The supplemental device according to claim 16, further comprising:
an alignment arrangement, wherein the alignment arrangement is configured to interact with a corresponding alignment feature of the injection device to couple the supplemental device to the injection device in one of a predetermined number of orientations relative to the injection device.

19. The supplemental device according to claim 16, further comprising:
a communication interface,
wherein the processor is configured to transmit data indicating the determined type of the injection device to an external computing device via the communication interface.

20. The supplemental device according to claim 16, wherein the sensor arrangement comprises a light source and a color sensor,
wherein the light source is arranged to illuminate a portion of the injection device when the supplemental device is coupled to the injection device,
wherein the color sensor is arranged to determine a color of the illuminated portion, and
wherein the processor is configured to determine the type of the injection device based on the determined color.

21. The supplemental device according to claim 16, wherein the sensor arrangement comprises a navigation switch movable between a plurality of positions,
wherein the navigation switch is arranged to be biased into a position of the plurality of positions by a biasing feature of the injection device when the supplemental device is coupled to the injection device,
wherein each position in the plurality of positions corresponds to a respective type of injection device, and
wherein the processor arrangement is configured to determine the type of the injection device based on the position of the navigation switch.

22. The supplemental device according to claim 16, wherein the sensor arrangement comprises a plurality of contacts arranged to contact a conductive track formed on a surface of the injection device when the supplemental device is coupled to the injection device,
wherein two or more contacts in the plurality of contacts are arranged to electrically couple one or more segments of the conductive track,
wherein the processor is configured to determine the type of the injection device by polling each of the contacts in the plurality of contacts.

23. The supplemental device according to claim 16, wherein the sensor arrangement comprises two pins arranged to contact a conductive track formed on a surface of the injection device when the supplemental device is coupled to the injection device,
wherein the processor is configured to determine the type of the injection device based on a determined resistance of the conductive track.

24. The supplemental device according to claim 16, wherein the sensor arrangement comprises a magnetic field sensor arranged to detect a magnetic field generated by the injection device when the supplemental device is coupled to the injection device,
wherein the processor is configured to determine the type of injection device based on the detected magnetic field.

25. The supplemental device according to claim 16, wherein the sensor arrangement comprises at least one proximity sensor arranged to determine a distance between the supplemental device and a respective identification feature of the injection device when the supplemental device is coupled to the injection device,
wherein the processor is configured to determine the type of the injection device based on the determined distance.

26. The supplemental device according to claim 25, wherein the proximity sensor comprises an infrared transceiver,
wherein the infrared transceiver is arranged to determine a depth of a hole arranged on a surface of the injection device when the supplemental device is coupled to the injection device,
wherein the processor is configured to determine the type of the injection device based on the determined depth of the hole.

27. The supplemental device according to claim 16, wherein the sensor arrangement comprises a first infrared sensor and a second infrared sensor,
wherein the first infrared sensor and second infrared sensor are both arranged to read an encoded track formed on the injection device as the supplemental device is moved relative to the injection device during coupling of the supplemental device to the injection device,
wherein the processor is configured to determine the type of the injection device based on the encoded track read using the first infrared sensor and second infrared sensor.

28. The supplemental device according to claim 16, wherein the sensor arrangement comprises a plurality of pins configured to form an electrical connection to a non-volatile memory in the injection device when the supplemental device is coupled to the injection device,
wherein the processor is configured to read data from the non-volatile memory when the supplemental device is coupled to the injection device, and
wherein the processor is configured to determine the type of the injection device based on the data read from the non-volatile memory.

29. A system comprising:
an injection device comprising an identification feature; and
a supplemental device comprising a sensor arrangement and a processor, wherein the processor is configured to cause the supplemental device to:
use the sensor arrangement to sense the identification feature of the injection device, and
determine a type of the injection device based on the sensed identification feature.

30. The system according to claim 29, wherein the injection device contains medicament.

31. The system according to claim 29, wherein the supplemental device further comprises a dose determination unit,
wherein the processor is further configured to cause the supplemental device to determine a dose of medicament contained in the injection device by using the dose determination unit, wherein the dose is determined based on the determined type of the injection device.

32. The system according to claim 29, wherein the supplemental device further comprises an alignment arrangement, wherein the alignment arrangement is configured to interact with a corresponding alignment feature of the injection device to couple the supplemental device to the injection device in one of a predetermined number of orientations relative to the injection device.

33. The system according to claim 29, wherein the supplemental device further comprises a communication interface,
wherein the processor is further configured to transmit data indicating the determined type of the injection device to an external computing device via the communication interface.

34. A method executed by a supplemental device, the method comprising:
sensing, by a sensor arrangement of the supplemental device, an identification feature of the injection device; and
determining, by a processor of the supplemental device, a type of the injection device based on the sensed identification feature.

35. The method according to claim 34, further comprising determining, by the supplemental device and based on the determined type of the injection device, a dose of medicament contained in the injection device.
